(19) United States
(12) Patent Application Publication
(10) Pub. No.: US 2021/0174925 A1
(43) Pub. Date: Jun. 10, 2021

(54) INTELLIGENT MONITORING, INTERACTIVE, AND WIRELESS INTERNET CONNECTED MEDICATION ADHERENCE, ANALYTICS, AND DATABASE SOLUTION

(71) Applicant: Daya Medicals, Inc. (Canada), Waterloo (CA)

(72) Inventor: RACA DAYA, West Palm Beach, FL (US)

(21) Appl. No.: 17/031,059

(22) Filed: Sep. 24, 2020

Related U.S. Application Data
(63) Continuation of application No. 16/045,407, filed on Jul. 25, 2018, now abandoned.
(60) Provisional application No. 62/536,776, filed on Jul. 25, 2017.

(51) Int. Cl. G16H 20/13; G16H 10/60; G16H 50/30; A61B 5/11; G16H 70/40; G06T 19/00; G16H 80/00; A61B 5/00; G16H 50/70; G16H 20/10; G16H 40/67

(52) U.S. Cl. CPC G16H 20/13; G16H 10/60; G16H 50/30; A61B 5/11; G16H 70/40; A61B 2562/0219; G16H 80/00; A61B 5/74; G16H 50/70; G16H 20/10; G16H 40/67; G06T 19/006

(57) ABSTRACT

Method and devices for a medication adherence platform including machine-learning analytics platform, and real-time pharmaceutical and consumer product fulfillment platform are provided. A device can comprise a sensor for sensing a medicine container or medicine, a database for storing patient related data, a computer readable medium for storing a patient treatment calendar, causing a patient's electronic device to transmit an alert based upon an event logged onto the patient treatment calendar determine medication adherence, storing data in the database, transmitting treatment-based information to the patient's device, and establishing an electronic communication channel between the patient and a healthcare professional.

### CROSS-REFERENCE TO RELATED APPLICATIONS

This application is a continuation of U.S. application Ser. No. 16/045,407, filed Jul. 25, 2018, which claims the benefit of U.S. Provisional Application Ser. No. 62/536,776, filed Jul. 25, 2017, the disclosure of each of which is hereby incorporated by reference in its entirety, including all figures, tables and drawings.

### BACKGROUND OF THE INVENTION

Medication adherence is a major issue facing the global healthcare system and economy. Patients failing to adhere to a treatment regimen often fail to take required dosages of medication or in other cases fail to take any medication at all.

Patients failing to adhere to treatment regimens often do so due to complex therapeutic regimens, patient confusion, and painful side effects. It is estimated that in the United States a lack of medication adherence costs the healthcare system an excess of $300 billion annually. These health-related costs are only expected to increase as the population ages. Almost of half of the adults in the US have one or more chronic conditions, with almost 22% of these adults using 3 or more prescriptions within the span of a month. Furthermore, 40% of senior hospital re-admissions are due to adverse drug events (ADEs).

Medication adherence has traditionally been defined in terms of a medication possession ratio (MPR) or whether a patient has picked up a prescription at the pharmacist in a one month time frame. However, this metric fails to account for lost medication, patient's failing to adhere to a treatment regimen, or patients distributing medication to a third party.

### BRIEF SUMMARY OF THE INVENTION

Arthur is a software platform that allows patients to use electronic devices to monitor and adhere to treatment regimens and connect with health care providers who can monitor and manage the patient's progress. The Arthur system can be used on multiple electronic devices including but not limited to mobile devices (for example, smart phones or tablets), wearable electronics (for example, watches, bracelets, or armbands), computing devices (for example, laptops or desktops), automated personal assistants and artificial intelligence assistants. Instead of relying on MPR, the Arthur system uses real-time medication adherence (RMA), which detects what medication a patient has taken, where the medication was taken, and when the medication was taken. This metric permits health care professionals to more accurately and remotely monitor a patient's adherence to a treatment regimen, and allows the Arthur system to predict and project a patient's medication adherence in the future.

The Arthur system can provide a patient with personalized medication schedules, provide audio, visual, augmented reality (AR) display instructions for medications, medical devices, and potential food suggestions to ingest along with the medication. The Arthur system can also cause a device to provide an audio, visual, or vibrational alert to indicate that a time to take medication is approaching, has arrived or has passed. The Arthur system can monitor a patient's adherence and notify health care professionals of adherence or non-adherence. The health care professionals have the opportunity to send additional notifications of non-adherence to the patient based upon the notifications.

The data collected through the Arthur system can be accessed by entities in a patient's healthcare ecosystem including but not limited to physicians, pharmacists, insurance professionals, food distributers, and medical device manufacturers or suppliers. The Arthur system also leverages Blockchain technology to provide secure and selectable transfer of information to and from a patient. The Arthur system includes functionalities to both request and receive information, products, or services from vendors. The Arthur system can also provide logistics and delivery solutions to deliver the information, products, or services to the patient.

### BRIEF DESCRIPTION OF THE DRAWINGS

FIG. 1 is a diagram illustrating the Arthur system.

FIG. 2 is an image of reports option provided to a user of the Arthur system.

FIG. 3 is an image of plot derived from data collected by the Arthur system.

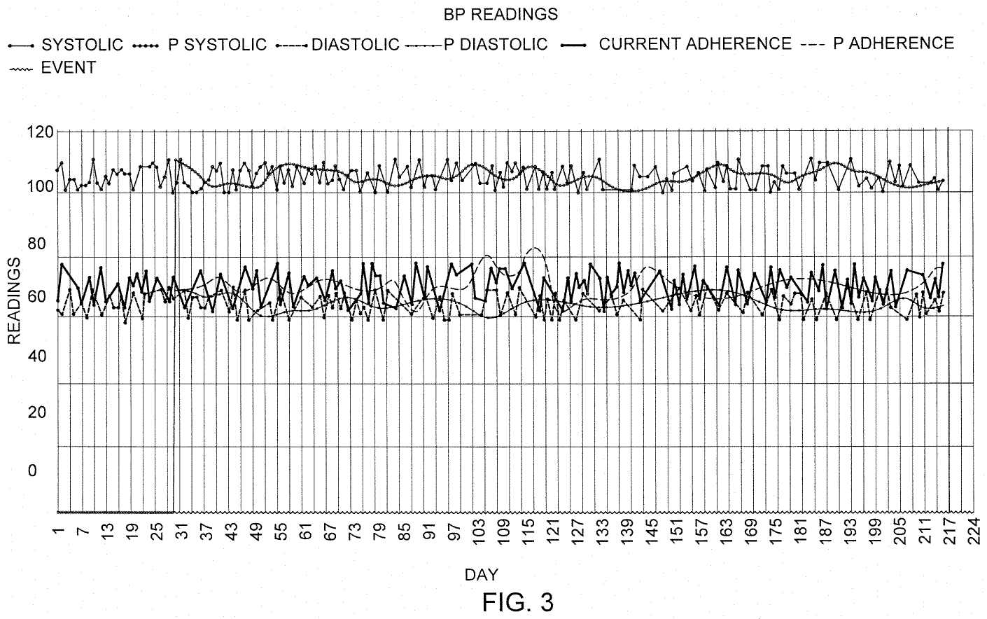

FIG. 4 is a set of images illustrating a medication container as seen by an augmented reality viewing device.

FIG. 5 is a block diagram of a method of determining medication adherence.

FIG. 6 is a block diagram of a method of determining medication adherence.

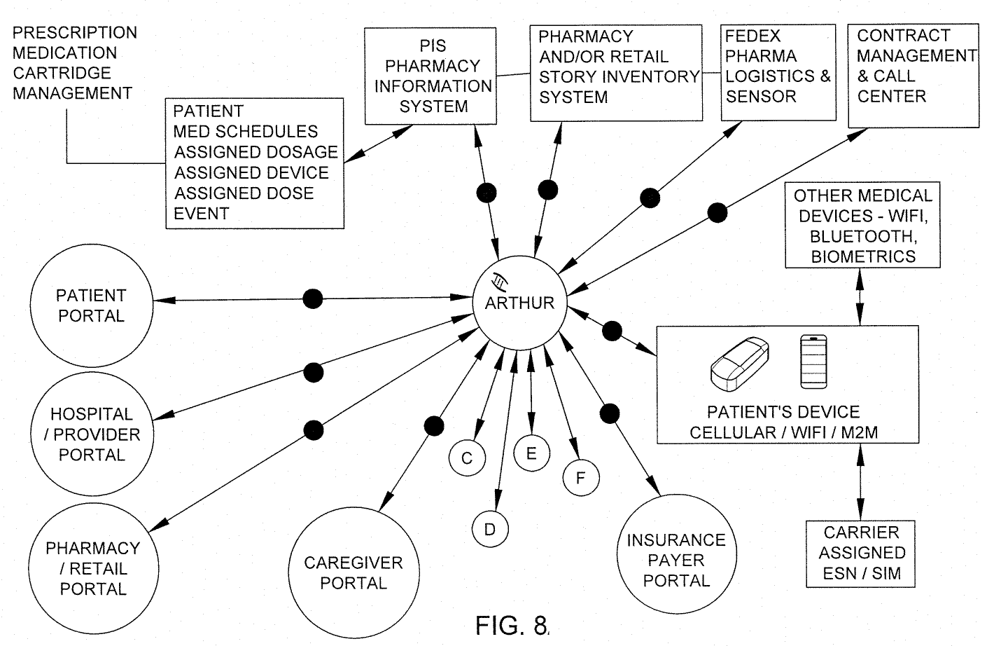

FIG. 7 is a block diagram of the Arthur system, according to an embodiment of the subject invention.

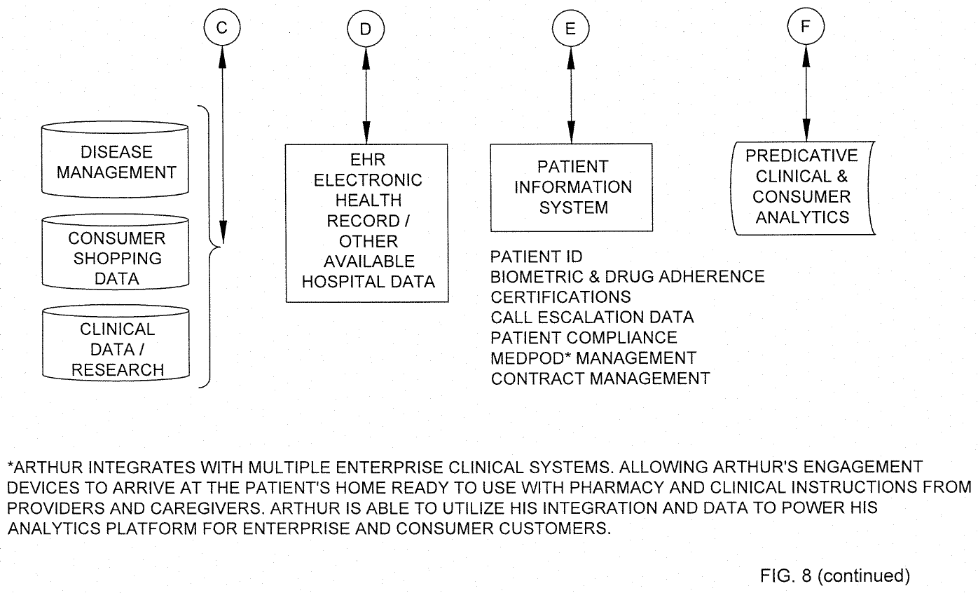

FIG. 8 is a block diagram of the Arthur system, according to an embodiment of the subject invention.

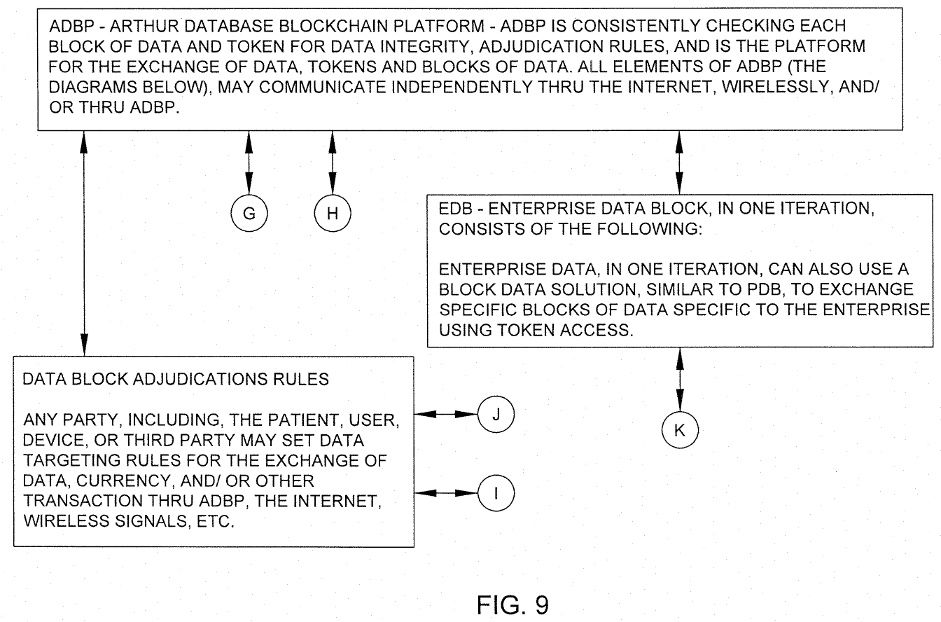

FIG. 9 is a block diagram of the Arthur system, according to an embodiment of the subject invention.

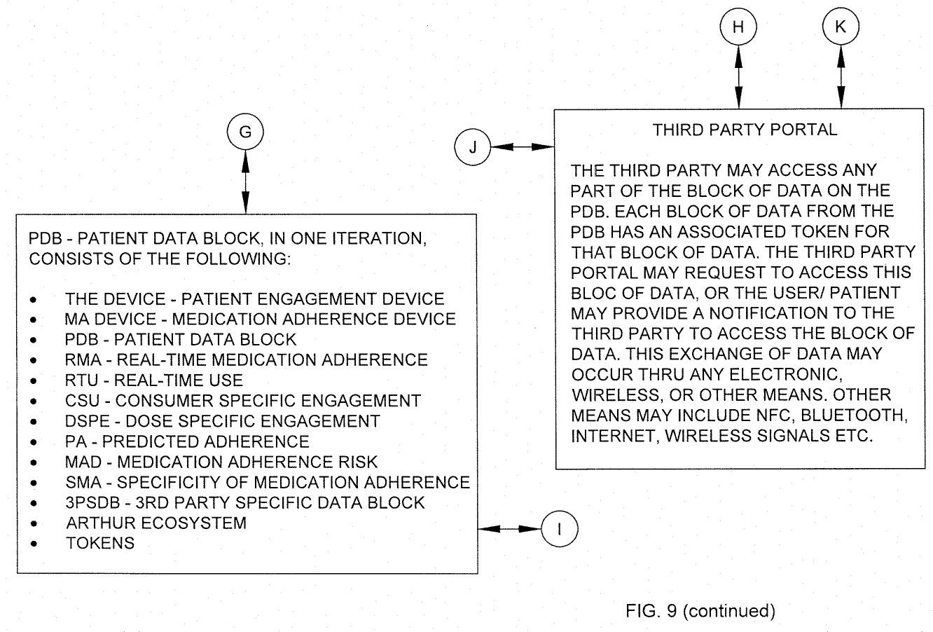

### DETAILED DISCLOSURE OF THE INVENTION

The following disclosure and exemplary embodiments are presented to enable one of ordinary skill in the art to make and use an interactive medical adherence system according to the subject invention. Various modifications to the embodiments will be readily apparent to those skilled in the art and the generic principles herein may be applied to other embodiments. Thus, the devices and methods related to the interactive medical adherence system are not intended to be limited to the embodiments shown, but are to be accorded the widest scope consistent with the principles and features described herein.

Arthur is a computing platform system comprising algorithms configured to operate on a mobile device, wearable electronics device, or computing device (hereinafter referred to as “the device”). The Arthur system comprises patient data including a treatment calendar, medication instructions, disease management information, treatment related products, and images and methods to securely exchange information between patients and members of the patient's healthcare ecosystem.

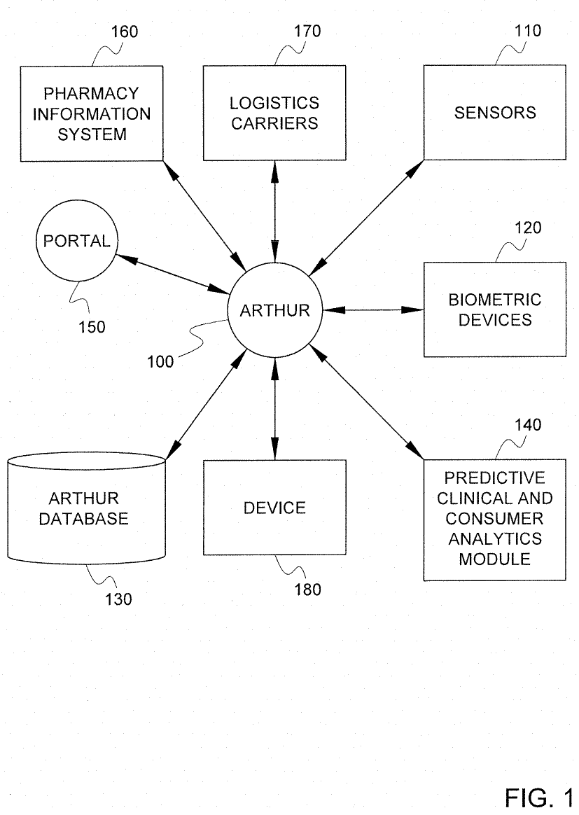

As seen in FIG. 1, the Arthur system 100 collects clinical and consumer data from a patient through sensors 110 (for example, an optical, pressure, thermo-, mass flow sensor, biometric, accelerometer, gyroscope, magnetometer or other sensor). Additionally, the Arthur system can collect data through biometric devices 120 (for example, a blood pressure cuff, glucometer, EKG, pulse oximeter, weight scale, or other biometric device). The data collected from the sensors 110, biometric devices 130, the patient, or various third parties can be stored in a database 130. The data stored on the database can be accessed by a predictive clinical and consumer analytics module 140. The module 140 can track a patient's medication adherence and provide predictive and projective analytics towards future adherence. Parties can access the Arthur system 100 through respective portals 150. These parties can include the patient, a hospital, health care professional, pharmacy, retailer, caregiver, or insurance provider. The Arthur system can be configured to restrict communication between specific parties and restrict input or retrieval of specific data or analytics stored in the database 130 to specific parties. The Arthur system can be in communication or integrated with a pharmacy information system 160. For example a patient can access the Arthur system 100 to directly communicate with a pharmacy regarding medication including request for refill or information regarding medication being taken. In turn the pharmacy can communicate with the patient and provide information regarding medication, alerts than medication needs to be refilled, or other medication related information. The Arthur system 100 can be connected and integrated with logistics carrier systems 170. A patient can communicate directly with a logistics carrier 170 through the Arthur system 100 to order, for example, prescriptions, retail products, or biometrics devices.

The Arthur system can be accessed by an electronic device 180, wherein the device can be used to transmit and receive patient information, including treatment information, pharmaceutical information, insurance information related to the treatment, related products information, and other notifications. A device 180 can comprise one or more modes of communication to reach a patient, healthcare provider, insurance provider, or medical device provider. In one embodiment, the device 180 can be used to effectuate video calls, conference calls, or telephone calls through the Arthur system 100 by accessing a network or internet connection. The Arthur system can further be configured to send e-mails to a patient. The Arthur system 100 can further be configured to communicate with a patient via social media, including permitting a third party or automated messaging system to transmit information and alerts to a patient's social media account.

The device 180 can comprise a memory device such as a Dynamic Random Access Memory (D-RAM), Static RAM (S-RAM), or other RAM or a flash memory, a storage device including a hard disk, a magneto-optical medium, an optical medium such as a CD-ROM, a digital versatile disk (DVDs), or BLU-RAY disc (BD), or other type of device for electronic data storage.

The device 180 can comprise a communication interface, for example, a communications port, a wired transceiver, a wireless transceiver, and/or a network card. The communication interface may be capable of communicating using technologies such as Ethernet, fiber optics, microwave, xDSL (Digital Subscriber Line), Wireless Local Area Network (WLAN) technology, wireless cellular technology, Bluetooth technology, NFC, RFID and/or any other appropriate technology.

The device 180 can comprise a display configured to communicate data including augmented reality (AR) imaging. The display device may operate using technology such as Video Graphics Array (VGA), Super VGA (S-VGA), Digital Visual Interface (DVI), High-Definition Multimedia Interface (HDMI), or other appropriate technology.

The device 180 can comprise a computer readable medium configured to perform any feature or any combination of features described herein. In such an instance, the memory device and/or the storage device may store instructions which, when executed by a processor, cause the processor to perform any feature or any combination of features described above as performed by the Arthur system 100. Alternatively or additionally, in such an instance, each or any of the features described above as performed by the Arthur system 100 may be performed by the processor in conjunction with the memory device, communication interface, display, and/or storage device.

In addition, the device 180 can include a display, a microphone, camera, and speakers. The device can be battery operated and charged using a USB, DC power cord, charging mat, or charged wirelessly charging.

A patient can access the Arthur system through a patient portal and interact with the Arthur system through a graphical user interface. In one embodiment the patient can be prompted to provide a username and password prior to receiving access to the Arthur system. Once a patient has access to the system, the patient can receive information regarding the patient's medical diagnosis, including condition, medication, and treatment plan.

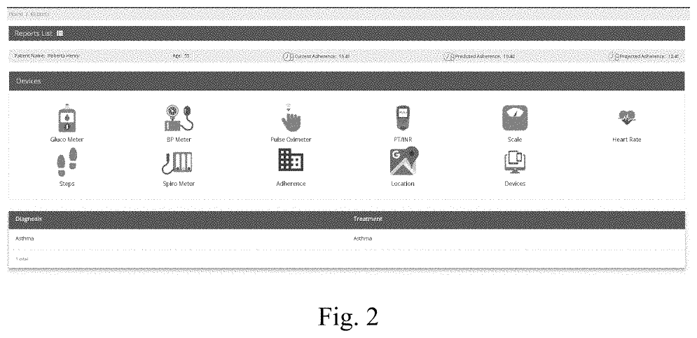

The patient can also navigate through different reports to monitor treatment progress, as seen in FIG. 2. The Arthur system can be integrated through a network with a plurality of biometric devices (including, but not limited to a blood pressure cuff, glucometer, EKG, pulse oximeter, etc.). The patient can either self operate these biometric devices or a third party can assist the patient. The readings determined by these biometric devices can be transmitted through the network to the Arthur system. A patient's biomarkers, including but not limited to heart rate, blood sugar level, and weight, can be stored and categorized by date, time, and location. In other embodiments, the data from a biometric device can be inputted directly from the patient or from a third party. In addition to inputting data from the plurality of biometric devices, the patient can access plots of the data points received from the various biometric devices over the course of a treatment plan, as seen in FIG. 2.

The device can comprise sensors and algorithms that allow the device to sense real time data and generate the reports based upon the data. The data and the reports can be stored on the device or transmitted to a third party, data acquisition unit, or database. The sensors can comprise an optical, pressure, thermo-, mass flow sensor, biometric, or other sensor. The Arthur system can record data actively with patient intervention or passively through sensors without the patient's initiation of the sensor. The sensed data can provide information regarding the medicine container, an object, the frequency of access to the medicine container or object, frequency of use, the temperature of the medicine container or object, or the volume over time of use of the medication in the medicine container or object. The device can be configured to be programmed directly by the patient or remotely by a third party.

The device can continuously collect real time data to create these reports and that can be transmitted to the Arthur database (AD). In one embodiment, the transmitted data can include information on patient's interaction with the device, an object, images, markers, use of and the amount of medication, real time location of the device, time of use, and amount of time of use of the device.

The Arthur system can be configured to receive and transmit this data through a network to various third parties including but not limited to a medical supplier, a pharmacy, a caregiver, logistics carrier, or a health care professional.

The Arthur system can provide an audio/visual, or vibrational alert to a patient related to events in treatment calendar. For example, the Arthur system can be configured to alert the patient to take a particular medication or that the time to take the medication has passed. The Arthur system can provide audio or visual instructions for using medications, or alerts from a health care professional or pharmaceutical company. The content of these directions can originate from a pharmaceutical company manufacturing the medication, the patient's family, the patient's healthcare professional, or any other approved source in the patient's medical ecosystem.

In one embodiment, a device can be configured to include circuitry, algorithms, and a light source to enable AR capabilities. The AR circuitry and algorithms can include a rendering engine module that permits the rendering of a virtual object on an existing object or background. In some embodiments, the device comprises a smart phone, a head mounted display (HMD), or a near eye display (NED).

The augmented reality (AR) circuitry and algorithms can also include a motion and positional tracking module. In one embodiment, motion tracking can be performed by an inertial measurement unit including an accelerometer, a gyroscope, or a magnetometer. Positional tracking can be performed by various methods including outside-in, inside-out, camera-based, marker-based, satellite-based, or other sensor-based method.

A patient can access the Arthur system through a device equipped with image capturing functionality to view a medicine container, including vials or organizers, an object, or a person (hereinafter collectively referred to as the “object”) with or without an AR image. In one embodiment, the Arthur system records instances of each AR image viewed by a patient. The Arthur system can then detect and store a time of the viewing and geolocation using GPS or other geolocation capability.

The AR image can provide medication instructions, disease management information, product for use by the patient, and/or images on the display of the device. In some embodiments, the AR image can be food items that a patient should take along with the medication. The AR images can be configured to be interactive and be selected by a patient to acknowledge medication adherence, initiate audio/video communication, and/or denote an action by the patient.

A patient's device can receive and transmit data through a computer-based network to provide alerts, collect environmental information, patient information, adherence information, biometrics information, or other information.

The Arthur system can be configured to operate with near field communication NFC circuitry. The device can be equipped with NFC circuitry. Near field communication (NFC) tags or other makers, can be placed on prescription vials, boxes, container, biometric devices, etc. Each NFC tag can be encoded with information related to the medication contained in the prescription vial, box, or container. In one embodiment, the upon activation of an NFC tag patient, the Arthur system will project on the device display an image that indicates that the wrong medication is being accessed. If, for example, a patient scans an NFC tag that does not correspond to the correct medication as indicated by the treatment calendar, the Arthur system can also notify the patient and others in the patient's healthcare ecosystem of the noncompliance and store the event in a database. Similarly, if the patient takes the correct medication, the system can log an entry that the patient has adhered to the treatment regimen. In one embodiment, the Arthur system records each instances of interaction with an NFC tag. The Arthur system can then detect and store a time and geolocation of the interaction using GPS or other geolocation capability.

The device can be equipped with sensors to detect the location of a marker, medication vial, medication container, biometric device (blood pressure cuff, glucometer, EKG, pulse oximeter, etc.) product, or object. The device can sense AR markered or markerless objects using computing devices with a camera, or in conjunction with external signals, sensors, or signal patterns.

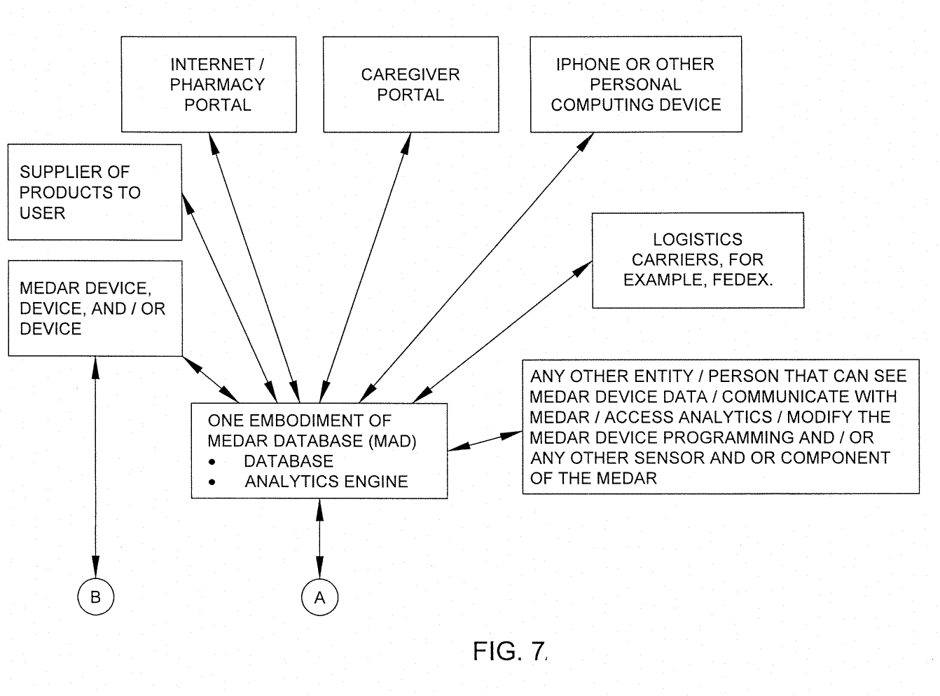

As seen in FIG. 4, an object 200, in this instance a box, can include an electronic device 210, in this instance a QR code and contain information that can be sensed by a sensor of the device. The electronic device can also comprise at least one of the following: electronic ink, radio frequency identification (RFID) tag, Bluetooth tag, NFC tag, QR codes, bar code, and/or other electronic device capable transmitting and/or receiving information from the electronic device. The electronic device 210 can also provide a location to render an AR image 220. The object can contain an additional object 200, in this instance, a medicine vial. The medicine vial can also include an electronic device 210 and provide a location for an AR image 220.

As seen in FIG. 4, in one embodiment, in order to determine RMA, the Arthur system can use the combination of the patient's treatment calendar with detection of an object, including but not limited to viewing an AR image on a medication vial, interacting with an NFC tag on a vial, or other detection of an object. A patient can detect an object S100 through interaction with an electronic device attached to the object. The system can log the time and location of the detection and then access the patient's treatment calendar S110. The patient's treatment calendar can be accessed before, simultaneously, or after detecting the object. The system can then compare the medication and time with the time and medication indicated on the patient's treatment calendar. The data indicating the patient's adherence or non-adherence can be stored in the database S130 and transmitted to a predictive clinical and consumer analytics module S140.

The Arthur system can provide audio, visual, or vibrational alerts through the device, for example an alert to prompt a patient, based upon the patient's treatment calendar, to use a biometric device, such as a blood pressure cuff, glucometer, or any other biometric sensor. The device can also provide audio or visual instructions for the biometric device. The biometric device can also be an object of which AR images are rendered upon to assist with directions and provide visual cues necessary for disease management.

The device can be used to manage prescriptions vials, medication boxes, and/or any other container of medication or medication directly. In one embodiment, the medicine container or objects comprise additional materials that identify the medicine containers or other objects.

In one embodiment, the Arthur system can render an AR image on the device display around the medicine container or object, for example a visual circle or virtual LED lighting providing a visual glowing of the medicinal container in preselected colors. In another embodiment, the Arthur system can be configured to present an image of arrow pointing towards the medicinal container or object to guide a patient. Once a medicine container medication is detected through sensing an AR marker, NFC tag or other means, the Arthur system can determine a location of the medicine container or medication using GPS or other geolocation technology.

The device can be configured to, at any time, detect the movement or removal of the medicine container or object. The device sensors or camera can use markers, weight, mass, or any other physical property, electronic device, radio frequency device to allow the device to detect/sense the scope of the movement, or amount of consumption of medication.

The device can be configured to include voice-activated and interactive speech recognition circuitry and algorithms permitting a patient to engage and communicate with the device. In another embodiment, the device is configured with pressure sensors to permit physical input from a patient. The Arthur system permits patients to make a video call, conference calls, telephonic calls, or send or receive SMS message through the Arthur system.

The AD can comprise machine analytics, computer learning, or artificial intelligence algorithms that generate data for third parties to offer other products, services, and/or health information to the patient through the device or other external method.

The data contained in the AD can be transmitted, stored, or view on the device or other electronic device. The Arthur system can be configured to transmit data from sensors to other electronic devices as to whether the patient is using the medicine or an object.

The device can contain built in wireless communication hardware and/or radios (for example, wireless internet, LTE, 3G, 4G, 5G, Wi-Fi, Bluetooth, and/or other wireless communication protocols to connect to other devices, wired/wireless biometric and vital signs sensors, databases, other sensors, other devices) touch sensitive viewing screens (LCD, LED, E-Ink, etc.), Global Position System sensors (GPS), thermometer, accelerometer, re-chargeable battery, NFC circuitry, RFID circuitry, microphone, speaker, keyboard, or other input/output mediums and sensors to allow the user to interact and transmit data from the device or the AD to a clinical staff person, physician, pharmacist, nurse, caregiver, friends, family, or any other entity. The transmission can be in the form of a video conference, phone call, instant message, text, social media, or other available method of communication through the Arthur system. The user can select particular data collected from the device and/or the AD to transmit to other parties.

The Arthur system can be configured to permit individuals to development and upload applications (i.e., third party development) onto the device or the AD to change or customize the user experience and functions of the device or the AD.

Third parties that are connected to the Arthur system can be provided permission to receive and transmit information from the device or the AD in real-time. In one example, the patient's pharmacist, health care professional, or insurance professional can transmit instructions to the patient based upon received data from the Arthur system. In another example, the transmitted instructions cause the audio, visual, vibrations alerts to occur on the device of a patient. If a patient misses a dosage time, dosage level, or otherwise indicates non-compliance with a treatment regimen, the Arthur system can transmit a notice to a third party through the device and create a log of compliance or non-compliance. The third party can then contact the patient's device directly via voice, video, chat, or through other communication means.

The Arthur system can transmit an auto-refill alert to a pharmacist or health care professional. For example, the device can be equipped with an image capturing feature that can capture an image of a medicine container to determine if the medication is above, at, or below a visual marker on the display of the device and based upon the image, transmit an auto-refill alert to the pharmacist or health care professional.

A patient can use the Arthur system to chat, video conference, place a telephone call to other users within a patient's ecosystem.

The Arthur system can also comprise software that allows the user to transmit only selected data to third parties or databases. The selected transmitted data can contain information including quantity, rate, type, location of the device, volume, biometric data, or other data. The Arthur system can also be configured to permit the patient to manually enter information relating to the following: notes, inputs, comments related to object being used; notes, inputs, or comments related to the mental and/or physical effects of the objects used; or notes, inputs, or comments related to the effect of the objects on the patient's condition.

In another embodiment, the Arthur system can be used in surgery for using, regulating, monitoring, and informing surgery staff of directions and use of surgical instruments. In another embodiment, engineers can use the Arthur system to give directions for use and monitor use of devices, instruments, and/or other equipment. In yet even another embodiment, logistics and shipping companies can use the Arthur system to monitor objects, monitor placement, movement, and/or any other characteristics of the objects before, during, or after shipping.

The Arthur system's interaction with an object creates a data metric known as Real-Time Use (RTU). When a patient interacts with or takes medication it is known as Real-Time Medication Adherence (RMA). RMA comprises of a situation in which the medication was taken using, when the medication was taken as determined by sensors, and a determination of what medication was taken as determined by sensors.

Based on the RTU, targeted messaging, including instructions, voice, video, and text communication can be transmitted to the patient. This targeted messaging is herein known as Consumer Specific Engagement (CSE). Patients can receive disease related information, patient demographic, medication, or other information to target specific services, clinical instructions, products, services. CSE can be effectuated by the patient being alerted to take their medication, otherwise known as Dose-Specific-Patient-Engagement (DSPE).

Another method of determining RMA is through the use of sensors to detect gestures that suggest adherence or non-adherence to a treatment regimen, and collect data to be transmitted to the AD. This is known herein as “gesture-based adherence” (GBA). The device can use sensors such as accelerators and gyroscopes to detect a patient's movement. A patient can be instructed to perform certain gestures and the predictive analysis (PA) module can be trained to predict adherence or non-adherence to a treatment regimen based upon the performed known gestures. The device can also include a photoplethysmogram (PPG) sensor(s) to detect light changes in the patient's skin to detect clinical markers of disease conditions or other biometric readings.

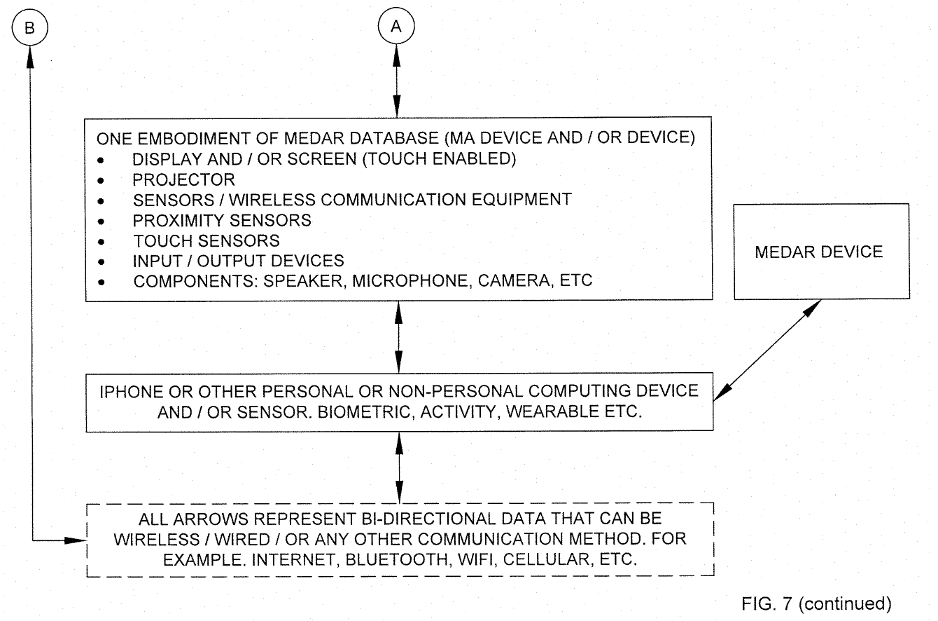

As seen in FIG. 5, in one embodiment, in order to determine GDA, the Arthur system uses the combination of the patient's treatment calendar with detection of gestures, including but not limited to viewing an AR image on a medication vial, interacting with an NFC tag on a vial, or other detection of an object. A patient can detect an object S200 through interaction with an electronic device attached to the object. The system can log the time and location of the detection and then detect a gesture S210 and access the patient's treatment calendar S220. The patient's treatment calendar can be accessed before, simultaneously, or after detecting the object or gesture. The system can then compare the medication and time with the time and medication indicated on the patient's treatment calendar S240. The system can additionally compare the detected gesture with a database if known gestures that indicates consumption S240. The data indicating the patient's adherence or non-adherence can be stored in the database S250 and transmitted to a predictive clinical and consumer analytics module S260.

Using a combination of the gyroscope, accelerometer, and PPG sensor(s) (described herein as GBA sensors) the Arthur system can collect data from the patient, which can be compared to a set of known gestures that reflect adherence with a treatment regimen. For example, gestures including a patient's wrist motion, motion during the opening of a medication vial, drinking motion, eating motion; can each indicate that the patient has put the medication in their mouth.

In one embodiment, in order to conserve energy, the GBA sensors are only activated after the patient initiates the AR or NFC technology. The GBA sensor data can be transmitted to the AD for additional analysis of medication adherence or non-adherence, and provide valuable data to clinicians, providers, or caregivers.

The GBA may be used independently of, in conjunction with, or to enhance the quality of data for the RTU, RMA, CSE, and DSPE; and provide additional data for engagement and intervention by the patient's health ecosystem. The RMA can also provide a metric of the GBA and stored as additional data in the AD. In another embodiment, the GBA sensor data (GSD) can be used to detect adherence to use of a medical devices, administration of other medical procedures, eating certain foods, drinking water, and executing a specific motion.

As the AD receives the GSD, the AD can create a database of respective GSD for patients. The AD can create new algorithms and uses existing algorithms to add additional accuracy and elevated prediction to correctly detect accurate medication adherence by the patient. If the patient's biometrics (e.g. weight, blood pressure, glucose, and/or any other clinical markers of a patient) are outside of normal levels after the patient takes their medication as measured by GSD, then the GSD receives additional weight in calculating future medication adherence based on GSD. In one embodiment, the algorithm may be structured with variables and weight in calculation (WC) as follows: Patient Alert for Medication (WC)+GSD (WC)+AR/NFC confirmation of vial (WC)+biometrics readings (WC)=Confirmation of Patient Medication Adherence (CPMA).

In the immediate aforementioned algorithm, not all variables need to present to calculate the CPMA, and there may be more variables used in predicting the CPMA, such as, activity of the patient, their GPS location, their consumer shopping habits, etc. In addition, another factor that can be included is the quality of the diet of the patient in conjunction with treatment regimen.

Using machine learning, the AD becomes more accurate at predicting CPMA as it collects more data from a patient's device, and the AD; and in one embodiment, may increase or reduce the WC value depending on the patient's disease condition, age, demographics, etc.

In one embodiment, the GDA and GSA may be utilized with a “blockchain” storage and process solution. For example, every patient may have their GDA, GSA, and CPMA added to a block of data, and given a specific token, value, or currency for tracking, analysis, and utilization by third parties.

The AD can employ block chain technology to store, transmit, and share information between the patient, pharmacist, physician, provide caregiver, wellness company, health payer/health insurance company, life insurance company, pharmaceutical company, consumer stores, an/or any other party. Each of these parties can have their own AD portal, application, or other to the AD's blockchain platform (ADBP) to engage with each other or the patient and share specific data sets that may be stored in the patient data block (PDB).

A medical provider may generate a prescription through their portal or by using a messaging service, such as email or text combined with or without a unique identifier that communicates with ADBP and the ADBP updates the PDB and assigns a specific token with that specific data block on the PDB. The medical provider can use a cell phone to call ADBP with a unique identifier, for example, with an issued token/identifier, their own phone number, or any other means for identification to update the PDB.

The ADBP may utilize its own platform or another existing third party platform for blockchain, such as Ethereum.

Data generated from the device, stored in the AD, or from the analytics of the AD can be stored, mined, and shared using specific permissions and token-based sharing protocols for each data set stored on the PDB. The patient can choose to share their specific prescription information on their PDB, a type of data block, with a pharmacy for one medication and or multiple medications. The patient would give their assigned token for that block of the ADBP's data to a pharmacy via an alphanumeric token through the ADBP or another unique identifier through the ADBP.

The pharmacy can use the token on their ADBP portal to access that specific prescription or specific set of data only. The data set may or may not include other patient specific information.

In one embodiment, the patient can choose to share their prescription, using the previously defined methodology, with a pharmaceutical company. The pharmaceutical company can send the prescription directly to the patient via a supply chain company or delivery company. In this previous example, the patient's health provider may issue/assign the prescription using the provider portal/engagement platform on the ADBP that updates the PDB. The patient can also execute an exchange of prescription information with a pharmacist or other provider by using a device to scan a QR code or detecting an NFC tag. This information can be transmitted directly to the pharmacist or provider or stored in a block of a blockchain.

In the immediate preceding example, this solution may function as a blockchain based e-prescribing solution.

The ADBP can transmit and receive information with supply chain and delivery companies. Any location, temperature, or other sensor data collected during the period of creation or process of the delivery from the sender to the patient can be updated to the ADBP, the patient PDB, and updates the data for that specific patient. This data may be shared using the ADBP's token and encryption methodology with any approved participant on the ADBPs portal, platform.

The Arthur system can use a predicative algorithm (PA) using data from the RTU, RMA, CSE, DSPE, the patient demographics (age, location, address, race, sex, or any patient specific information), medications taken or to be taken, disease states of the patient, consumer shopping behavior, and/or any other specific patient data. Arthur's PA can allow hospitals, payers, insurance companies, employers, unions, pharmaceutical companies, pharmacy benefit management companies, or any other entity or individual the ability to predict whether a patient or individual will be adherent to a specific medication or clinical protocol.

In another embodiment, using derived analytics from the Arthur system, the AD will use the PA to predict future adherence, and prompt events based on declining or inclining biometric data. Events may be calls to the patients, interventions from the healthcare ecosystem, emails, or any other means of communication. Hence, the Arthur system can provide data in regards to predicted adherence, projected adherence, and current adherence.

Arthur's PA can be used by insurance companies or any other entities to power a Value-Based Insurance Design (VBID)/Payment Model (VBID—is a term of art used to describe entities in the medical industry using both patient data and third party data to price products and services, or reward clinical entities for providing/generating value on clinical outcomes of patients). Arthur's PA can be performed before or after a treatment regimen begins. Arthur's PA will give each factor a specific WC in its predication process that can be pre-set using available clinical studies, any other data, and/or set manually. Arthur's PA can also use data captured by the Arthur system, the patient, an electronic device, wireless biometrics, medication adherence information captured by the device or entered by the patient, available hospital information of the patient, available claims data, or any other available database. As more patient specific data is added to the AD, the PA can predict a more specific indication of medication adherence or clinical outcomes of the patient, and a more specific patient profile for use by the medical ecosystem, consumer stores, or other entities.

In one embodiment the PA take as inputs one more of the following factors in calculation: patient demographics (WC)+patient medication (WC)+patient disease state (WC)+patient biometrics data (WC)+patient device medication adherence information (WC)+patient consumer shopping data (WC)=medication adherence risk, and/or clinical risks of the patient

The data derived from the RTU, RMA, CSE, MAD, PA, or DSPE may be used to generate patient specific consumer or medical product (“product”) placement on the display of the device.

The patient's location can be determined using the patient's IP address, location of the patient's device, location of the device or other locating mechanism.

Same-day, next-day, and/or other logistics or delivery solutions can be utilized for delivery of the requested product by automatically receiving the location of the device. The patient can also choose to receive or pick-up the product at a pre-determined or self-reported location. In another embodiment, the patient can use an image capturing device capture to capture an image of the medication prescription, a written medical procedure, medication(s), consumer product, or other prescription. This image can be transmitted through the Arthur system to a pharmacy, hospital, and/or any other third party. The transmitted prescription in the image can be delivered by relying on the same processes described above for real-time fulfillment using the patient's location, or self-reported address.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processer reads and executes the code and/or data stored on a computer-readable medium, the computer system and/or processer performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A computer-based medical adherence system, comprising:
a sensor that senses a medication container or medication;
a database that stores patient related data;
a computer readable medium comprising stored instructions that when executed cause at least one processor to:
electronically store a patient treatment calendar;
cause a patient's electronic device to transmit an alert based upon an event logged onto the patient treatment calendar;
determine medication adherence by a patient by comparing detection of a medication container or medication to an event logged on the patient treatment calendar;
store data collected from the sensor and from determination of medication adherence in the database;
transmit treatment-based information to the patient's device; and
establish an electronic communication channel between the patient and a healthcare professional.

2. The computer-based medical adherence system of claim 1, wherein the system further comprises a device that is a mobile device, a wearable electronic device, a computing device, an automated personal assistant, or an artificial intelligence assistant for accessing the system.

3. The computer-based medical adherence system of claim 2, wherein the device comprises augmented reality circuitry that renders an augmented image on an existing object.

4. The computer-based medical adherence system of claim 3, wherein the augmented image is at least one of instructions for using medication, a food item to be ingested along with medication, or a notification from a third party.

5. The computer-based medical adherence system of claim 1, wherein the alert is an audio, visual, or vibrational alert.

6. The computer-based medical adherence system of claim 1, wherein the sensor comprises near field communication circuitry, radio frequency identification circuitry, a QR code scanner, a barcode scanner, an optical scanner, or a pressure-sensing scanner.

7. The computer-based medical adherence system of claim 1, wherein the system further comprises a predictive analytics module, wherein the predictive analytics module is configured to use predictive algorithms to predict or project future medication adherence, and wherein the predictive analytics module is further configured to transmit and receive data from the database.

8. The computer-based medical adherence system claim 1, wherein the computer readable medium is further configured to permit the healthcare professional to access data collected from the sensor.

9. The computer-based medical adherence system claim 1, wherein the computer readable medium is further configured to permit a pharmacy information system to receive data from and transmit data to the database.

10. The computer-based medical adherence system claim 1, wherein the computer readable medium is further configured to establish an electronic communication channel between the patient and a pharmacy, a logistics carrier, or a freight carrier.

11. The computer-based medical adherence system claim 10, wherein the computer readable medium is further configured to store and separate data into a block of a blockchain, and wherein the computer readable medium is further configured to selectively permit access to the data contained in the block by the healthcare professional, the pharmacy, the logistics carrier, or the freight carrier.

12. A computer-based medical adherence system, comprising:
a sensor that senses a medication container or medication;
a sensor that senses physical gestures of a patient;
a database that stores patient related data;
a computer readable medium comprising stored instructions that when executed cause at least one processor to:
electronically store a patient treatment calendar;
cause a patient's electronic device to transmit an alert based upon an event logged onto the patient treatment calendar;
determine medication adherence by a patient by
comparing detection of a medication container or medication to a medication-based event on the patient treatment calendar; and
comparing a physical gesture of the patient to a database of known physical gestures that indicate consumption of the medication;
store data collected from the sensor and the determination of medication adherence in the database;
transmit treatment based information to the patient's device; and
establish an electronic communication channel between the patient and a healthcare professional.

13. The computer-based medical adherence system claim 12, wherein the sensor that senses the physical gestures of the patient comprises a gyroscope, an accelerometer, or a magnetometer.

14. The computer-based medical adherence system of claim 13, wherein the device further comprises augmented reality circuitry that renders an augmented image on an existing object.

15. The computer-based medical adherence system of claim 13, wherein the augmented image is instructions for using medication, a food item to be ingested along with medication, or a notification from a third party.

16. The computer-based medical adherence system of claim 12, wherein the system further comprises a predictive analytics module, wherein the predictive analytics module is configured to use predictive algorithms to predict or project future medication adherence, wherein the predictive analytics module is further configured transmit and receive data from the database.

17. A computer-based method for determining medication adherence, the method comprising:
sensing, by a sensor, a medication container or medication;
detecting, a time and location of sensing the medication container or the medication;
accessing an electronic patient treatment calendar;
determining medication adherence by comparing the sensed medication container or medication with a time and medication indicated on an event on the electronic patient calendar;
storing data collected from the sensor and determination of medication adherence in a database; and
analyzing the data stored on the database with a predictive algorithm to predict and project future medication adherence.

18. The computer-based method for determining medication adherence of claim 17, the method further comprising:
detecting, by a sensor, that a medication supply is depleted;
establishing an electronic communication channel between the patient and a pharmacy; and
requesting, through the electronic communication channel, a refill of the medication supply.

19. The computer-based method for determining medication adherence of claim 17, the method further comprising:
establishing an electronic communication channel between the patient and a logistics carrier or freight carrier;
communicating with the logistics carrier or freight carrier, through the electronic communication channel, to manage a time and a place of delivery of the medication supply.

20. The computer-based method for determining medication adherence of claim 17, the method comprising:
determining that the patient is not adherent based on an event logged on the electronic patient treatment calendar; and
causing a patient's device to transmit an audio, visual, or vibrational alert.
